(19) United States
(12) Patent Application Publication
(10) Pub. No.: US 2003/0105438 A1
(43) Pub. Date: Jun. 5, 2003

(54) Apparatus and method for an ostomy harness

(76) Inventor: Clara S. Hostetler, Hartville, OH (US)

Correspondence Address: Heather M. Barnes, Brouse McDowell, 500 First National Tower, 106 S. Main Street, Akron, OH, 44308-1471, US

(21) Appl. No.: 10/309,841

(22) Filed: Dec. 4, 2002

Related U.S. Application Data
(60) Provisional application No. 60/338,584, filed on Dec. 4, 2001.

(51) Int. Cl. A61F 5/44; A61M 1/00

(52) U.S. Cl. 604/345; 604/540

(57) ABSTRACT

An ostomy harness comprises an ostomy bag cover that is attached to a belt. The bag cover is selectively and adjustably attached to the belt, such that the stoma may be relieved of the weight from the ostomy bag. Suspenders may be attached to the belt. Further, the ostomy harness may be made of a soft material that is comfortable to the user.

This utility patent application claims priority from a provisional patent application having serial No. 60/338,584 filed on Dec. 4, 2001. Inventorship remains the same.

### 1. BACKGROUND OF THE INVENTION

A. Field of Invention

This invention pertains to the art of methods and apparatuses for colostomy harness, and more specifically, to a methods and apparatuses for a colostomy harness that is adjustable to relieve pressure and stress from a stoma.

B. Description of the Related Art

Because of disease or other pathological conditions, or due to inflammation or surgery, portions or all of a person's intestines are sometimes rendered temporarily or permanently inoperative. In these instances, a surgical procedure known as an ostomy is performed, in which a passageway or stoma is made through the stomach wall and skin, typically through the abdominal wall. A portion of the intestine is rerouted and surgically connected to the stoma such that waste material can exit the body.

As is well known in the art, the term “ostomy” describes all types of surgical procedures wherein a passageway is formed through the skin and a portion of the intestine is connected thereto. If the large intestine or colon is connected to the passageway, the procedure is known as a “colostomy”. When the small intestine is involved, the procedure is known as an “ileostomy.” Both of these procedures require use of an ostomy bag, which is worn on the body of the user and is in communication with the stoma to collect waste emitted by the body. A wide variety of ostomy bags and supports have been developed in the prior art.

One problem in the art is undue weight and stress being placed on the area surrounding the stoma. Colostomy bag covers known in the art simply comprise a belt. While these harnesses suit their intended purpose, undue stress is still placed on the stoma area as the colostomy bag fills with waste. As the weight increases in the colostomy bag, the belt may cause sagging around the stoma area. Some belt designs have compensated for this problem by making the belt wider in height. Nevertheless, it is believed that user comfort is compromised.

Because of the foregoing problems and other, there is a need in the art of colostomy bag covers to provide a colostomy bag harness that is comfortable to the user and yet provides support around the stoma area. The present invention provides methods and apparatuses for protecting ostomy bags. The difficulties inherit in the art are therefore overcome in a way that is simple and efficient, while providing better and more advantageous results.

### II. Summary of the Invention

An ostomy harness comprises an ostomy bag cover that is attached to a belt. The bag cover is selectively and adjustably attached to the belt, such that the stoma may be relieved of the weight from the ostomy bag. Suspenders may be attached to the belt. Further, the ostomy harness may be made of a soft material that is comfortable to the user.

It is an object of the present invention to provide an ostomy bag harness that keeps weight off of the stoma when walking or traveling.

It is yet another object of the present invention to provide an ostomy bag harness that is more suitable for travel and is more comfortable.

Still, yet another object of the present invention to provide an ostomy bag harness comprising an extra soft plastic around the stomach bag or other absorbent material inside the harness to prevent embarrassing seepage or leaks.

Another object of the present invention is to provide an ostomy bag harness that is washable.

Further, another object of the present invention is to provide suspenders attached to the belt to provide added support to the ostomy bag harness.

Another object of the present invention is to provide an ostomy bag harness that is constructed of a soft and comfortable material, such as but not limited to, soft fleece or acetate and polyester.

Yet, another object of the present invention is to provide an ostomy bag harness that hides a colostomy bag, as well as, provides easy access to empty the colostomy bag if needed.

Still yet, another object of the present invention is to provide an ostomy bag that is easy for the patient to open and close.

Another object of the present invention is to provide an apparatus that is an ostomy bag harness comprising a first supporting means, a second supporting means operatively connected to the first supporting means; and, a cover operatively connected to the first supporting means, the cover adapted to support an associated colostomy bag, wherein the first supporting means supports the colostomy bag in a substantially horizontal direction and the second supporting means supports the colostomy bag in a substantially vertical direction.

Further, another object of the present invention is to provide an apparatus, wherein the first supporting means is a belt.

It is another object of the present invention to provide an apparatus, wherein the second supporting means is at least one suspender.

Still yet, another object of the present invention is to provide an apparatus, wherein the cover is adjustable and the cover comprises a first end; and, a second end oppositely disposed from the first end, the second end adapted to selectively engage the belt, wherein the first end and the second end forming a pouch when the second end is selectively engaged to the belt, the pouch having a hole defined therein for selectively receiving the colostomy bag.

It is yet another object of the present invention to provide an apparatus, wherein the adjustable cover is a single piece.

Further, another object of the present invention is to provide an apparatus, wherein the suspender is adjustable.

Yet another object of the present invention to provide an apparatus, wherein the suspender is selectively positioned on the first supporting means.

Another object of the present invention is to provide an apparatus, wherein the second supporting means is adapted to maintain the position of the first supporting means with respect to an associated stoma.

It is yet another object of the present invention to provide a colostomy bag harness, comprising a belt, at least one suspender operatively connected to the belt, and an adjustable cover adapted to enclose an associated colostomy bag, the adjustable cover adapted to selectively position the colostomy bag relative to an associated stoma to reduce tension on the skin surrounding the stoma.

Still yet, another object of the present invention is to provide a colostomy bag harness, wherein the adjustable cover comprises a first end; and, a second end oppositely disposed from the first end, the second end adapted to selectively engage the belt, wherein the first end and the second end forming a pouch when the second end is selectively engaged to the belt, the pouch having a hole defined therein for selectively receiving the colostomy bag.

Another object of the present invention is to provide a colostomy bag harness, wherein the suspender is adapted to maintain the position of the belt with respect to an associated stoma.

Another object of the present invention is to provide a colostomy bag harness, wherein the colostomy bag is made from cotton.

Further, another object of the present invention is to provide a method for using a colostomy bag harness comprising the steps of:

providing a belt, at least one suspender operatively connected to the belt; and,

an adjustable cover adapted to enclose an associated colostomy bag, the adjustable cover having a first end attached to the belt and a second end, the adjustable cover adapted to selectively position the colostomy bag relative to an associated stoma to reduce tension on the skin surrounding the stoma, the cover having a hole defined therein to selectively receive a colostomy bag;

positioning the belt about an associated user's waist;

positioning the suspender about the user's shoulders;

positioning the colostomy bag through the hole;

attaching the second end of the cover to the belt; and,

positioning the colostomy bag relative to an associated stoma to reduce tension on the skin surrounding the stoma.

Still, another object of the present invention is to provide a colostomy bag harness, wherein the cover encloses the colostomy bag to define a pouch, the pouch having a length L, the method further comprising the steps of:

increasing the amount of matter in the colostomy bag;

repositioning the second end of the cover on the belt so as to decrease the length L of the pouch.

Another object of the present invention is to provide a colostomy bag harness wherein the cover encloses the colostomy bag to define a pouch, the pouch having a length L, the method further comprising the step of respositioning the second end of the cover on the belt so as to increase the length of the pouch.

Yet, another object of the present invention is to provide a method, further comprising the step of supporting the belt in a substantially vertical direction, wherein the suspender provides support in the vertical direction.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein:

FIG. 1 is a front view of the present invention showing the optional suspenders attached to the belt.

FIG. 2 is a back view of the colostomy bag cover.

FIG. 3 is a front view of the colostomy bag cover.

FIG. 4 is a perspective view of the colostomy bag cover in a partially opened position.

FIG. 5 is a rear view of the belt showing the belt ends attached together.

### IV. DESCRIPTION OF THE PREFERRED EMBODIMENT

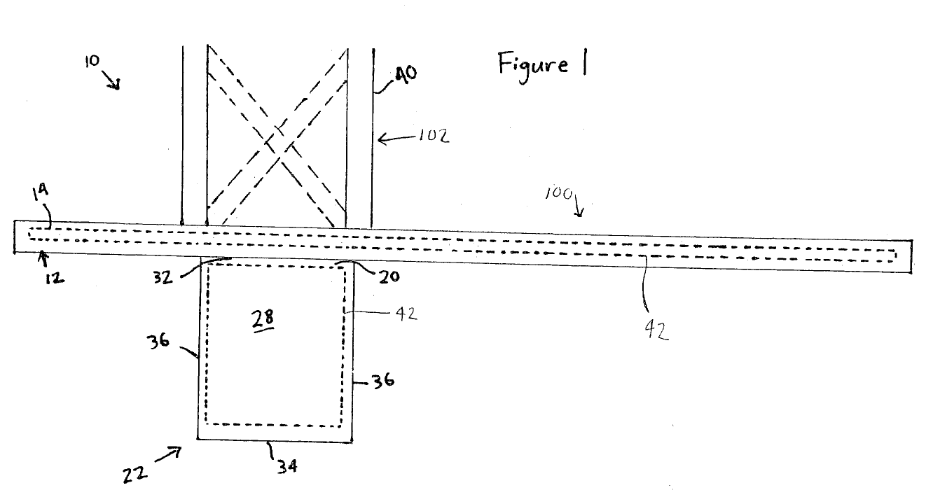

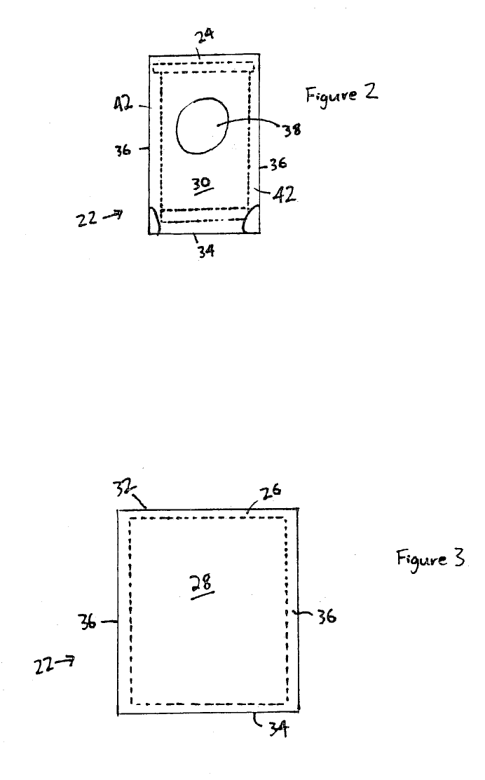

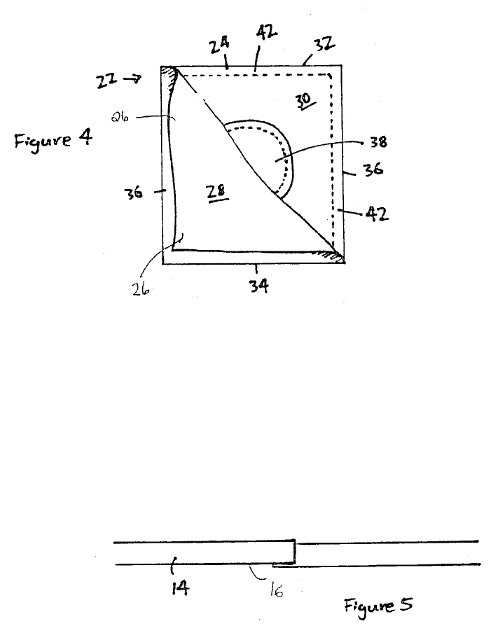

Referring now to the drawings wherein the showings are for purposes of illustrating a preferred embodiment of the invention only and not for purposes of limiting the same, FIGS. 1-5 show the present invention. FIGS. 1-4 show an ostomy bag harness 10. The ostomy bag harness 10 comprises a first supporting means 100, which may take the form of a belt 12. Second supporting means 102, which may take the form of a suspender 40, is operatively connecting to the first supporting means 100. The ostomy bag harness 10 further comprises a cover 22 operatively connected to the first supporting means 100. The cover 22 is adapted to support an associated colostomy bag 10. The first supporting means 100 supports the colostomy bag 10 in a substantially horizontal direction and the second supporting means 102 supports the colostomy bag 10 in a substantially vertical direction.

The belt 12 has a first surface 14 and a second surface 16, wherein the second surface 16 contacts the user's skin or clothes. The belt 12 further comprises a first end 18 and second end 20, which may be secured together through Velcro 42, as best seen in FIG. 5. As shown in FIG. 5, the second surface 16 contacts the first surface 14 for secure attachment. The Velcro 42 on the belt 12 may extend the length of the belt 12 to provide for easy adjustment. Of course, the first and second ends 20 may be attached through any other means chosen in accordance with sound engineering judgment, including without limitations, buckles, snaps, buttons, or tie strings.

With reference to FIGS. 1-4, a bag cover 22 is operatively connected to the belt 12. The bag cover 22 comprises a first end 24 and a second end 26 oppositely disposed from the first end 24. The first end 24 may be sewn to the belt 12. However, the first end 24 may be attached to the belt 12 through any means chosen with sound engineering judgment. The second end 26 is adapted to be selectively and adjustably attached to the belt 12 through Velcro 42, or other easily attachable means, such as snaps, buttons, or other equivalent fastener. When the second end 26 is attached to the belt 12, a pouch member is formed having a top 32, a bottom 34 and sides 36. The bag cover 22 should be of sufficient size to accommodate an ostomy bag (not shown). For example, when the second end 26 is attached to the belt, the bag width may be approximately six inches, and the height may be approximately nine inches. Further, the height may range from seven to eleven inches as the bag cover 22 is adjusted.

A back portion 30 of the bag cover 22 comprises a circular opening 38 for the ostomy bag to be positioned therethrough. It should be noted that the shape of the opening 38 is not important. What is important is that the ostomy bag may easily fit through the opening 38 and yet have sufficient support. A front portion 28 of the bag cover 22 covers the front of the ostomy bag.

As shown in the FIGURES, Velcro 42 is used around the perimeter of bag cover 22. This enables the bag to be adjusted in a vertical direction to accommodate the size of the ostomy bag disposed therein. As an ostomy bag fills with waste material and excrement, the second end 26 of the bag cover 22 may be repositioned at a higher position on the belt so that the weight on the stoma is reduced. This is more comfortable for the user and also prevents unsightly bulges.

As shown in FIG. 1, suspenders 40 may be attached to the belt 12 through sewing or through Velcro 42. This is particularly advantageous for a person who has undergone an ileostomy procedure because the belt 12 would be worn high around the user's waist. Rather than using Velcro 42, any other fastener may be used that is easy to open and close, including without limitations, snaps or buttons. It is also contemplated to be within the scope of the present invention to provide an adjustable suspender 40. Further, the suspender may be selectively positioned on the first supporting means 100.

In order to use the present invention, an ostomy bag is positioned within the opening 38 of the bag cover 22. The belt 12 is adjusted and secured around the user's waist. The second end 20 is positioned appropriately on the belt 12 so that the stoma does not have any excess weight or stress placed on it. When the ostomy bag needs to be emptied, the second end 20 is removed from the belt such that the bag cover 22 is opened. The ostomy bag can easily be emptied from the bottom. It does not need to be removed through the opening. After it is emptied, the second end 20 can then again be repositioned on the belt 12. If the suspenders 40 need to be utilized, they are attached to the belt 12. The suspenders then may be adjusted to comfortably fit the user.

The material of the belt 12 should be soft and comfortable to the user. Such material may be chosen in accordance with sound engineering judgment, but may include without limitation fleece, acetate, or polyester.

The preferred embodiments have been described, hereinabove. It will be apparent to those skilled in the art that the above methods may incorporate changes and modifications without departing from the general scope of this invention. It is intended to include all such modifications and alterations in so far as they come within the scope of the appended claims or the equivalents thereof.

Having thus described the invention, it is now claimed:

What is claimed is:

1. An apparatus, comprising:
a first supporting means;
a second supporting means operatively connected to said first supporting means; and,
a cover operatively connected to said first supporting means, said cover adapted to support an associated colostomy bag, wherein said first supporting means supports the colostomy bag in a substantially horizontal direction and said second supporting means supports said colostomy bag in a substantially vertical direction.

2. The apparatus of claim 1, wherein said first supporting means is a belt.

3. The apparatus of claim 1, wherein said second supporting means is at least one suspender.

4. The apparatus of claim 1, wherein said cover is adjustable, said cover comprising:
a first end; and,
a second end oppositely disposed from said first end, said second end adapted to selectively engage said belt, wherein said first end and said second end forming a pouch when said second end is selectively engaged to said belt, said pouch having a hole defined therein for selectively receiving the colostomy bag.

5. The apparatus of claim 4, wherein said adjustable cover is a single piece.

6. The apparatus of claim 3, wherein said suspender is adjustable.

7. The apparatus of claim 3, wherein said suspender is selectively positioned on said first supporting means.

8. The apparatus of claim 1, wherein said second supporting means is adapted to maintain the position of said first supporting means with respect to an associated stoma.

9. A colostomy bag harness, comprising:
a belt;
at least one suspender operatively connected to said belt; and,
an adjustable cover adapted to enclose an associated colostomy bag, said adjustable cover adapted to selectively position the colostomy bag relative to an associated stoma to reduce tension on the skin surrounding the stoma.

10. The colostomy bag harness of claim 9, wherein said adjustable cover comprises:
a first end; and,
a second end oppositely disposed from said first end, said second end adapted to selectively engage said belt, wherein said first end and said second end forming a pouch when said second end is selectively engaged to said belt, said pouch having a hole defined therein for selectively receiving the colostomy bag.

11. The colostomy bag of claim 9, wherein said suspender is adapted to maintain the position of said belt with respect to an associated stoma.

12. The colostomy bag of claim 9, wherein said colostomy bag is made from cotton.

13. The colostomy bag of claim 9, wherein said adjustable cover comprises:
a first end; and,
a second end oppositely disposed from said first end, said second end adapted to selectively engage said belt, wherein said first end and said second end define a pouch when said second end is selectively engaged to said belt, said pouch having a hole defined therein for selectively receiving the colostomy bag, wherein said suspender is adapted to maintain the position of said belt with respect to an associated stoma, wherein said colostomy bag is made from cotton, said belt adapted to support said colostomy bag in a substantially horizontal direction, said suspender adapted to support said belt and said colostomy bag in a substantially vertical direction, said suspender being elastic.

14. A method for using a colostomy bag harness comprising the steps of:
providing a belt, at least one suspender operatively connected to said belt; and,
an adjustable cover adapted to enclose an associated colostomy bag, said adjustable cover having a first end attached to said belt and a second end, said adjustable cover adapted to selectively position the colostomy bag relative to an associated stoma to reduce tension on the skin surrounding the stoma, said cover having a hole defined therein to selectively receive a colostomy bag;
positioning said belt about an associated user's waist;
positioning said suspender about the user's shoulders;
positioning said colostomy bag through said hole;
attaching said second end of said cover to said belt; and,
positioning the colostomy bag relative to an associated stoma to reduce tension on the skin surrounding the stoma.

15. The method of claim 14, wherein the cover encloses the colostomy bag to define a pouch, said pouch having a length L, the method further comprising the steps of:
increasing the amount of matter in the colostomy bag;
repositioning the second end of the cover on said belt so as to decrease the length L of the pouch.

16. The method of claim 14, wherein the cover encloses the colostomy bag to define a pouch, said pouch having a length L, the method further comprising the step of:
respositioning the second end of the cover on said belt so as to increase the length of the pouch.

17. The method of claim 14, further comprising the step of: supporting said belt in a substantially vertical direction, wherein said suspender provides support in the vertical direction.
